SEQUENCE LISTING (22 sequences)

SEQ ID NO: 1 - DNA, 1255 bp, Homo sapiens
  CDS (31)..(1029)
agtatcctcccaaaggtgacactggaagcaatgaacaccacagtgatgcaaggcttcaacagatctgagcggtgccccagagacactcggatagtacagctggtattcccagccctctacacagtggttttcttgaccggcatcctgctgaatactttggctctgtgggtgtttgttcacatccccagctcctccaccttcatcatctacctcaaaaacactttggtggccgacttgataatgacactcatgcttcctttcaaaatcctctctgactcacacctggcaccctggcagctcagagcttttgtgtgtcgtttttcttcggtgatattttatgagaccatgtatgtgggcatcgtgctgttagggctcatagcctttgacagattcctcaagatcatcagacctttgagaaatatttttctaaaaaaacctgtttttgcaaaaacggtctcaatcttcatctggttctttttgttcttcatctccctgccaaatatgatcttgagcaacaaggaagcaacaccatcgtctgtgaaaaagtgtgcttccttaaaggggcctctggggctgaaatggcatcaaatggtaaataacatatgccagtgtattttctggactgtttttatcctaatgcttgtgttttatgtggttattgcaaaaaaagtatatgattcttatagaaagtccaaaagtaaggacagaaaaaacaacaaaaagctggaaggcaaagtatttgttgtcgtggctgtcttctttgtgtgttttgctccatttcattttgccagagttccatatactcacagtcaaaccaacaataagactgactgtagactgcaaaatcaactgtttattgctaaagaaacaactctctttttggcagcaactaacatttgtatggatcccttaatatacatattcttatgtaaaaaattcacagaaaagctaccatgtatgcaagggagaaagaccacagcatcaagccaagaaaatcatagcagtcagacagacaacataaccttaggctgacaactgtacatagggttaacttctatttattgatgagacttccgtagataatgtggaaatcaaatttaaccaagaaaaaaagattggaacaaatgctcccttacattttattatcctggtgtacagaaaagattatataaaatttaaatccacatagatctattcataagctgaatgaaccattactaagagaatgcaacaggatacaaatggccactagagg

SEQ ID NO: 2 - protein, 333 aa, Homo sapiens
MNTTVMQGFNRSERCPRDTRIVQLVFPALYTVVFLTGILLNTLALWVFVHIPSSSTFIIYLKNTLVADLIMTLMLPFKILSDSHLAPWQLRAFVCRFSSVIFYETMYVGIVLLGLIAFDRFLKIIRPLRNIFLKKPVFAKTVSIFIWFFLFFISLPNMILSNKEATPSSVKKCASLKGPLGLKWHQMVNNICQCIFWTVFILMLVFYVVIAKKVYDSYRKSKSKDRKNNKKLEGKVFVVVAVFFVCFAPFHFARVPYTHSQTNNKTDCRLQNQLFIAKETTLFLAATNICMDPLIYIFLCKKFTEKLPCMQGRKTTASSQENHSSQTDNITLG

SEQ ID NO: 3 - DNA, 28 bp, Artificial
  Artificially synthesized primer sequence
cagaggcccctttaaggaagcacacttt

SEQ ID NO: 4 - DNA, 26 bp, Artificial
  Artificially synthesized primer sequence
tcacagacgatggtgttgcttccttg

SEQ ID NO: 5 - DNA, 26 bp, Artificial
  Artificially synthesized primer sequence
gagaccatgtatgtgggcatcgtgct

SEQ ID NO: 6 - DNA, 28 bp, Artificial
  Artificially synthesized primer sequence
agcgctcatagcctttgacagattcctc

SEQ ID NO: 7 - DNA, 556 bp, Homo sapiens
ctgaactaatgactgccgccataagaagacagagagaactgagtatcctcccaaaggtgacactggaagcaatgaacaccacagtgatgcaaggcttcaacagatctgagcggtgccccagagacactcggatagtacagctggtattcccagccctctacacagtggttttcttgaccggcatcctgctgaatactttggctctgtgggtgtttgttcacatccccagctcctccaccttcatcatctacctcaaaaacactttggtggccgacttgataatgacactcatgcttcctttcaaaatcctctctgactcacacctggcaccctggcagctcagagcttttgtgtgtcgtttttcttcggtgatattttatgagaccatgtatgtgggcatcgtgctgttagggctcatagcctttgacagattcctcaagatcatcagacctttgagaaatatttttctaaaaaaacctgtttttgcaaaaacggtctcaatcttcatctggttctttttgttcttcatctccctgccaaatatgatcttgagcaa

SEQ ID NO: 8 - DNA, 913 bp, Homo sapiens
aagatcatcagacctttgagaaatatttttctaaaaaaacctgtttttgcaaaaacggtctcaatcttcatctggttctttttgttcttcatctccctgccaaatatgatcttgagcaacaaggaagcaacaccatcgtctgtgaaaaagtgtgcttccttaaaggggcctctggggctgaaatggcatcaaatggtaaataacatatgccagtttattttctggactgtttttatcctaatgcttgtgttttatgtggttattgcaaaaaaagtatatgattcttatagaaagtccaaaagtaaggacagaaaaaacaacaaaaagctggaaggcaaagtatttgttgtcgtggctgtcttctttgtgtgttttgctccatttcattttgccagagttccatatactcacagtcaaaccaacaataagactgactgtagactgcaaaatcaactgtttattgctaaagaaacaactctctttttggcagcaactaacatttgtatggatcccttaatatacatattcttatgtaaaaaattcacagaaaagctaccatgtatgcaagggagaaagaccacagcatcaagccaagaaaatcatagcagtcagacagacaacataaccttaggctgacaactgtacatagggttaacttctatttattgatgagacttccgtagataatgtggaaatcaaatttaaccaagaaaaaaagattggaacaaatgctcccttacattttattatcctggtgtacagaaaagattatataaaatttaaatccacatagatctattcataagctgaatgaaccattactaagagaatgcaacaggatacaaatggccactagaggtcattatttctttctttctttttttttctagaattcagcggccgctgaattcta

SEQ ID NO: 9 - DNA, 28 bp, Artificial
  Artificially synthesized primer sequence
agtatcctcccaaaggtgacactggaag

SEQ ID NO: 10 - DNA, 28 bp, Artificial
  Artificially synthesized primer sequence
cctctagtggccatttgtatcctgttgc

SEQ ID NO: 11 - DNA, 32 bp, Artificial
  Artificially synthesized primer sequence
gcggccgcatgaacaccacagtgatgcaaggc

SEQ ID NO: 12 - DNA, 32 bp, Artificial
  Artificially synthesized primer sequence
cggtccgcctaaggttatgttgtctgtctgac

SEQ ID NO: 13 - DNA, 22 bp, Artificial
  Artificially synthesized primer sequence
gctttcggcaccatgggctgca

SEQ ID NO: 14 - DNA, 25 bp, Artificial
  Artificially synthesized primer sequence
tcacactgcaggaccatctgtcaca

SEQ ID NO: 15 - DNA, 31 bp, Artificial
  Artificially synthesized primer sequence
tctagacggaccgatgggctgcacgctgagc

SEQ ID NO: 16 - DNA, 33 bp, Artificial
  Artificially synthesized primer sequence
ggatccttaaaagagaccaatatcttttagatt

SEQ ID NO: 17 - DNA, 23 bp, Artificial
  Artificially synthesized primer sequence
gagcgaggcgggagggtgtgtgt

SEQ ID NO: 18 - DNA, 23 bp, Artificial
  Artificially synthesized primer sequence
gaagggcagggcgggtgtctagc

SEQ ID NO: 19 - DNA, 31 bp, Artificial
  Artificially synthesized primer sequence
ttcggaccgatgactctggagtccatcatgg

SEQ ID NO: 20 - DNA, 33 bp, Artificial
  Artificially synthesized primer sequence
agggatccttagaccagattgtactccttcagg

SEQ ID NO: 21 - DNA, 34 bp, Artificial
  Artificially synthesized primer sequence
gccggaccgatgggctgcctcgggaacagtaaga

SEQ ID NO: 22 - DNA, 33 bp, Artificial
  Artificially synthesized primer sequence
gctctagaatttgggggttcccttcttagagca

(19) United States
(12) Patent Application Publication
(10) Pub. No.: US 2003/0059878 A1
(43) Pub. Date: Mar. 27, 2003

(54) Novel purinoceptor and gene thereof

(75) Inventors: Tetsuo Onuki, Satte-shi (JP); Yutaka Koguchi, Saitama-shi (JP); Naoki Tsuda, Tsuchiura-shi (JP)

Correspondence Address: BROWDY AND NEIMARK, P.L.L.C., 624 NINTH STREET, NW, SUITE 300, WASHINGTON, DC, 20001-5303, US

(73) Assignee: TANABE SEIYAKU CO., LTD, Osaka (JP)

(21) Appl. No.: 10/189,576

(22) Filed: Jul. 8, 2002

Related U.S. Application Data
(60) Provisional application No. 60/306,433, filed on Jul. 20, 2001.

(30) Foreign Application Priority Data
Jul. 20, 2001 (JP) 2001-206914

(51) Int. Cl. C07K 14/705; C12P 21/02; C12N 5/06; C07H 21/04

(52) U.S. Cl. 435/69.1; 435/320.1; 435/325; 530/350; 530/358; 536/23.5

(57) ABSTRACT

This invention provides a novel G protein-coupled receptor, i.e. a purinoceptor (P2Y receptor), and a gene thereof. It further provides a novel method for screening, identifying or characterizing a ligand and agonist or antagonist. That is, this invention is concerned with a polypeptide having the function or activity of a purinoceptor which is selected from the following (A), (B) and (C):

(A) a polypeptide having an amino acid sequence of SEQ ID NO: 2,

(B) a polypeptide having an amino acid sequence shown by SEQ ID NO: 2 in which one or plural amino acids are deleted, substituted or added,

(C) a polypeptide encoded by a nucleic acid or a complement thereof, said nucleic acid being hybridizable under stringent conditions to the nucleic acid having the nucleotide sequence of SEQ ID NO: 1,

and a nucleic acid encoding the polypeptide, a recombinant vector or a host cells which contain said nucleic acid, and further concerned with a method for detecting the function or activity of the polypeptide by using them, or a method for screening or identifying a ligand, agonist or antagonist to the polypeptide.

### TECHNICAL FIELD

The present invention relates a novel receptor (purinoceptor) protein and a gene thereof, and further a method for screening a medical candidate compound using them.

### TECHNICAL BACKGROUND

It is known that physiological substances such as many neurotransmitters, hormones, autacoid control the function of biobody via a specific receptor protein which exists on the surface of cells. Many of these receptors are coupled with GTP-binding protein (hereinafter, referred to as “G protein”) and can transduce the information by activation thereof. Accordingly, these receptors are called as G protein-coupled receptor. It is also known that the G protein-coupled receptors have a common structure including 7 transmembrane regions.

G protein is a trimer consisting of α, β and γ subunits and is present in an inactive form (GDP-bound form) where those subunits associate in the ground state. When the G protein-coupled receptor is stimulated by a ligand, the inactive G protein (GDP-bound form) is changed to the active form (GTP-bound form) and thereby it dissociates into the GTP-bound a subunit (Gα) and β/γ subunit complex (Gβγ). Then, the GTP-bound Gα (or occasionally Gαγ) controls an effector such as adenylate cyclase, phospholipase C, etc. to transmit the signals.

G protein includes various kinds of Gα, and depending on the kinds of Gα, it controls different kinds of effectors.

Generally, G protein-coupled receptor activates a specific G protein and through said activated G protein specific information is transmitted into cells.

As the G protein-coupled receptors there have hitherto been known α- and β-adrenaline receptor, muscarinic acetyl-choline receptor, adenosine receptor, angiotensin receptor, endothelin receptor, gonadotropin-releasing factor receptor, H₁- and H₂-histamine receptor, dopamine receptor, metabolic glutamic acid receptor, somatostatin receptor, etc. All of them have important roles as a target for physiologically active substances in the biobody. It is also very interesting that many of known medicaments are a ligand, or antagonist or agonist for G protein-coupled receptors.

From this viewpoint, a great attention has been given to G protein-coupled receptors as a target for development of medicaments. It has highly been desired to find a new G protein-coupled receptor, to identify a ligand therefor, and to find a method for screening or identifying an agonist or antagonist thereof, because those are effective for screening candidates of new medicaments.

As to purinoceptor, it has been known as follows.

Purinoceptor is classified into P1 receptor having high affinity to adenosine (it is also called as “adenosine receptor”) and P2 receptor having a high affinity to ATP (it is also called as “ATP receptor”). The P2 receptor is further classified into an ionic channel type receptor where the receptor protein per se composes ionic channel (P2X and P2Z type receptors) and a metabolic control type receptor which exhibits the function by activating G protein (P2Y, P2U and P2T type receptors). Among them, the P1 receptor and the metabolic control type P2 receptor are a sort of G protein-coupled receptors (cf. Harada et al., “IGAKU-NO-AYUMI” (March of Medical Science), Vol. 181, pp. 199-202, 1997).

These receptors are further classified into various subtypes depending on the kinds of ligands or affinity thereof. For example, among the metabolic control type P2 receptors, those for which natural ligand is ATP and ADP are designated as P2Y receptor. Besides, the receptors for which natural ligand is ATP but not ADP are designated as P2T receptor, and those for which natural ligand is ATP and UTP are designated as P2U receptor (cf. King et al., Trends in Pharmacological Sciences, Vol. 19, pp. 506-514, 1998; Harden et al., Annu. Rev. Pharmacol. Toxicol., Vol. 35, pp. 541-579, 1995).

### DISCLOSURE OF INVENTION

An object of the present invention is to provide a novel G protein-coupled receptor and a gene thereof. More particularly, it is to provide a novel purinoceptor (P2Y receptor) and a gene thereof. It further provides a novel method for screening, identifying or characterizing a ligand and modulator (agonist or antagonist). Other objects of the invention will be apparent from the following description.

The present inventors have isolated a full length cDNA coding for a new G protein-coupled receptor, and further have succeeded to express the receptor protein in cells by a genetic engineering technology. Further, the inventors have identified the ligand therefor and have found that the receptor is purinoceptor (P2Y receptor). Then, the present invention has been accomplished.

That is, the present invention relates to a polypeptide having the function or activity of a purinoceptor which is selected from the following (A), (B) and (C):

(A) a polypeptide having an amino acid sequence of SEQ ID NO: 2,

(B) a polypeptide having an amino acid sequence shown by SEQ ID NO: 2 in which one or plural amino acids are deleted, substituted or added,

(C) a polypeptide encoded by a nucleic acid or a complement thereof, said nucleic acid being hybridizable under stringent conditions to the nucleic acid having the nucleotide sequence of SEQ ID NO: 1.

The present invention further relates to a nucleic acid encoding the polypeptides, a recombinant vector or a host cell which contains said nucleic acid.

Furthermore, the present invention relates to a method for detecting the function or activity of the polypeptides by using the polypeptides, or a method for modulating (enhancing or depressing) the function or activity of the polypeptides by using them, or a method for screening or identifying a ligand or modulator (agonist or antagonist) of the polypeptides.

The SEQ ID No: 1 shown in sequence listing disclosed hereinafter is the nucleotide sequence of a human cDNA of a gene of the receptor protein (designated as “TG0022 protein”) (including the full length of the coding region), which was isolated by the present inventors.

SEQ ID NO: 2 is an amino acid sequence of the receptor protein (TG0022 protein) encoded by the cDNA.

The receptor protein of the present invention is a G protein-coupled receptor protein and has a function (biological activity) as a purinoceptor, more particularly, can specifically bind to ATP, ADP, or their analogues [for example, alkylthio-adenine nucleotide derivatives such as 2-methylthioadenosine 5′-triphosphate (designated as “2MeSATP”) and 2-methylthioadenosine 5′-diphosphate (designated as “2MeSADP”), etc.]. The receptor protein of the present invention is stimulated by the specific binding, and thereby the intracellular signal transduction is induced.

The natural agonists for the receptor protein of the present invention are ATP or ADP. Besides, the analogues such as 2MeSATP or 2MeSADP also function as the agonist.

Moreover, when the receptor protein of the present invention is simulated by the ligand (agonist), it activates G protein which belongs to Gᵢ subfamily. Aiming at α subunit, by stimulation of the receptor protein with the ligand (agonist), G_{iα} (α subunit of G protein belonging to Gᵢ subfamily, e.g. G_{iαl}) is changed to the active form (which has GTP binding property), and owing to activation of G protein, signal is intracellularly transduced.

As mentioned above, the receptor protein (polypeptide) of the present invention has the function of the following (i), (ii) and (iii):

(i) specific binding properties to ATP, ADP or their analogues,

(ii) induction of intracellular signal transduction (e.g. change of concentration of Ca²⁺, change of concentration of cAMP, activation of phospholipase C, change of pH value and change of concentration of K⁺) based on stimulation by ATP, ADP or their analogues,

(iii) activation of G protein (a subunit of G protein belonging to Gᵢ subfamily) based on stimulation by ATP, ADP or their analogues.

The ligands for the receptor protein of the present invention are ATP or ADP as mentioned above, and further the receptor protein of the present invention has the homologous amino acid sequence with those of known receptor proteins [e.g., P2Y₁ receptor (NCBI accession no. XP_{—}003033, PIR/SWISS-PROT accession no. P47900), P2Y₂ receptor (NCBI accession no. XP_{—}006367, PIR/SWISS-PROT accession no. P41231), P2Y₄ receptor (NCBI accession no. XP_{—}010396, PIR/SWISS-PROT accession no. P51582), P2Y₆ receptor (NCBI accession no. XP_{—}006366, PIR/SWISS-PROT accession no. Q15077), P2Y₁₁ receptor (NCBI accession no. NP_{—}002557), P2Y12 receptor (NCBI accession no. NP_{—}073625)), and hence, it is assumed that the receptor of the present invention is one of P2Y receptors.

In the present invention, “ligand” means a compound specifically binding to the receptor protein. The ligand includes both of natural compounds and artificially synthesized compounds.

The “agonist” means a compound which has an ability of inducing of intracellular signal transduction by stimulating the receptor protein by means of specifically binding thereto, or the like. The “antagonist” means a compound which has an ability to depress the activity of the compound having an ability of inducing intracellular signal transduction by stimulating the receptor protein.

### BRIEF DESCRIPTION OF DRAWINGS

FIG. 1 shows an amino acid sequence, nucleotide sequence and the assumed 7 transmembrane region (the underlined region) of TG0022.

FIG. 2 shows an expression map of TG0022 gene in each tissue or cell in human (obtained by dot blotting), wherein round mark means the tissue or cell in which the expression of a gene at the level of mRNA was observed.

FIG. 3 is a schematic figure showing outline of the structure of a fusion protein of TG0022 protein and various kinds of G protein.

FIG. 4 shows effects of the test compounds on the amount of specifically binding of GTPγS to the membrane fraction containing the fusion protein of TG0022 protein and various kinds of G protein. In FIG. 4(A), the test compound was 2MeSATP (2-methylthioadenosine 5′-triphosphate). In FIG. 4(B), the test compound was 2MeSADP (2-methylthioadenosine 5′-diphosphate). In FIG. 4(C), the test compound was ATP. In FIG. 4(D), the test compound was ADP. In these FIG. 4(A) to FIG. 4(D), the specific binding amount is indicated by the relative value (% of control) to the binding amount in control where no test compound was used. Besides, in FIG. 4(A) to FIG. 4(D), ┌◯G_{iαl} (³⁵¹Cys→Ile)┘ means the data of the membrane fraction containing a fusion protein of TG002 protein and G_{iαl} (³⁵¹Cys→Ile). ┌ΔG_{qα}┘ means the data of the membrane fraction containing a fusion protein of TG002 protein and G_{qα}. ┌□_{sαL}┘ means the data of the membrane fraction containing a fusion protein of TG002 protein and G_{sαL}.

### BEST MODE FOR CARRYING OUT THE INVENTION

The protein or polypeptide of the present invention include a polypeptide having an amino acid sequence of SEQ ID NO: 2, and also include a polypeptide having an amino acid sequence shown by SEQ ID NO: 2 in which one or plural amino acids are deleted, substituted or added. The deletion, substitution or addition of amino acid(s) may be in such a degree that it does not lose the function (biological activity) of a purinoceptor (P2Y receptor), and the number of amino acid(s) to be deleted, substituted or added may usually be in the range of 1 to about 80 amino acids, preferably 1 to about 60 amino acids, more preferably 1 to about 45 amino acids, further preferably 1 to about 30 amino acids, specifically preferably 1 to about 15 amino acids.

That is, the protein or polypeptide of the present invention includes the polypeptide having an amino acid sequence of SEQ ID NO: 2 and further a polypeptide having one or more conservative amino acid substitutions compared with the polypeptide having an amino acid sequence of SEQ ID NO: 2.

Such a protein or polypeptide includes naturally occurring variant proteins or polypeptides and also includes artificially modified mutant proteins or peptides and further proteins or peptides derived from other living species. That is, the protein or polypeptide of the present invention includes conservative substitution variants and naturally occurring allelic variants of the polypeptide having an amino acid sequence of SEQ ID NO: 2.

Such proteins or polypeptides have a homology in the amino acid level, e.g. usually about 75% or more, preferably about 80% or more, more preferably about 85% or more, further preferably about 90% or more, especially preferably about 95% or more homology in the amino acid sequence of SEQ ID NO: 2.

The nucleic acid (DNA or RNA) of the present invention includes a nucleic acid having a nucleotide sequence of SEQ ID NO: 1, and further a nucleic acid being hybridizable under stringent conditions (preferably under highly stringent conditions) to the nucleic acid having the nucleotide sequence of SEQ ID NO: 1 or a complement thereof (i.e. a nucleic acid having a complemental sequence). Such a hybridizable nucleic acid may be those encoding a protein or peptide having a function (biological activity) as a purinoceptor (P2Y receptor).

Such nucleic acids have usually about 70% or more, preferably about 80% or more, more preferably about 85% or more, further preferably about 90% or more, especially preferably about 95% or more homology in the nucleotide sequence of SEQ ID NO: 1. Such genes or nucleic acids include naturally occurring mutant genes, artificially modified mutant genes, homologous genes derived from other living species (orthologues), etc.

The hybridization under stringent conditions is usually carried out under the conditions: in a hybridization solution having a salt concentration of 6×SSC or similar thereto, at a temperature of 50 to 60° C. for about 16 hours, and optionally subjecting to pre-washing with a solution having a salt concentration of 6×SSC or similar thereto and then subjecting to washing with a solution having a salt concentration of 1×SSC or similar thereto. When it is carried out under higher stringent conditions (highly stringent conditions), the washing in the above procedure is carried out with a solution having a salt concentration of 0.1×SSC or similar thereto.

The nucleic acid of the present invention can be obtained by screening the genes originated from the tissues or cells of mammals. The mammals include non-human animals such as dogs, cows, horses, goats, sheep, monkeys, pigs, rabbits, rats, and mice, as well as humans. Among them, human genes are preferable in view of using for research and development of medicaments useful for treatment of diseases in human being.

The nucleic acids of the present invention can be obtained by utilizing the information of sequences disclosed in this description (SEQ ID NO: 1 in the sequence listing disclosed hereinafter). For instance, a primer and a probe are designed based on the information on the disclosed nucleotide sequence, and then by optionally combining a PCR (polymerase chain reaction) technology, a colony hybridization method, and a plaque hybridization method using them, the desired nucleic acid is isolated from DNA library.

For example, cDNA is prepared from mRNA isolated from mammalian cells or tissues, and by using it as a template, a cDNA fragment is obtained by PCR method. By using the cDNA fragment thus obtained as a probe, cDNA library is screened by a colony hybridization method or a plaque hybridization method to obtain a full length cDNA. Besides, a genomic DNA is isolated by screening genomic DNA library. Further, by screening DNA libraries of other mammals, homologous genes from other living species (orthologues) may be obtained.

The cDNA library and genomic DNA library can be obtained by the method disclosed in a literature, for example in “Molecualr Cloning” (Sambrook, J., Fritsch, E. F. and Maniatis, T., Cold Spring Harbor Laboratory Press, 1989). Alternatively, when a library is commercially available, it may be used.

By determining the nucleotide sequence of the cDNA thus obtained, the coding region encoding the protein of the gene product can be determined, and thereby, the amino acid sequence of the protein can be determined.

The protein or polypeptide of the present invention can be produced by overexpression by an usual recombinant DNA technique. It may also be produced by expressing in the form of a fusion protein with other protein or peptide.

The cells where the protein or peptide of the present invention is overexpressed can be obtained, for example, by the following method.

Firstly, a DNA encoding the protein or peptide of the present invention is inserted into a vector so that it is linked downstream of an appropriate promoter, thereby preparing an expression vector, and then, the obtained expression vector is introduced into a host cell.

The expression system (host-vector system) includes, for example, expression system of bacteria, yeasts, insectile cells, and mammalian cells. Among them, insectile cells (e.g. Spodoptera frugiperda SF9, SF21, etc.) and mammalian cells (e.g. monkey COS-7 cells, Chinese hamster CHO cells, human HeLa cells, etc.) are preferably used as the host in order to obtain a protein reserving well the desired function.

The promoter useful for expressing the protein or polypeptide includes SV40 promoter, LTR promoter, Elongation 1α promoter, etc. in case of mammalian cell system, and polyhedrin promoter, etc. in case of insectile cell system.

The vector includes retrovirus vector, Papillomavirus vector, vaccinia virus vector, SV40 vector, etc. in case of mammalian cell system, and Baculovirus vector, etc. in case of insectile cell system.

The DNA encoding the protein or polypeptide of the present invention may be a cDNA corresponding to the naturally occurring mRNA (e.g. the cDNA having a nucleotide sequence as shown in SEQ ID NO: 1), but it is not limited thereto. It may be prepared by designing a DNA corresponding to the amino acid sequence of the desired protein of the present invention. In this method, as the codon coding for each one amino acid, there are one to six codons, and among which any codon may freely be chosen, but it is preferable to design a DNA having the best expression efficiency under taking into consideration the frequency of the codons appeared in the host used. The DNA having the desired nucleotide sequence to be thus designed may be obtained by a method of chemical synthesis of DNA, by a method of preparing cDNA fragments and binding them, or by a method of modifying a part of the nucleotide sequence. The artificial modification of a part of the nucleotide sequence or mutation may be done by a PCR method or site specific mutagenesis (cf. Mark et al., Proceedings of National Academy of Sciences, Vol. 81, pp. 5662-5666, 1984), etc., using a primer comprising a synthetic oligonucleotide coding the desired modification.

The protein or polypeptide of the present invention can be isolated and purified from the cultured product of the cells, into which the expression vector is introduced, by a conventional purification method, for example by appropriately combining salting-out with inorganic bases, fractionation and precipitation in an organic solvent, ion exchange column chromatography, affinity column chromatography, gel filtration, etc.

By overexpression of the protein or polypeptide of the present invention, the function or activity of the protein or polypeptide can be enhanced.

The nucleic acids (oligonucleotides, polynucleotide) or complements thereof which are hybridizable with the nucleic acid of the present invention under the stringent conditions can be used as a probe for detecting the gene of the present invention. In order to modify (e.g. depress) the expression of the present gene, they may be used, for example, as an antisense oligonucleotide, ribozyme, decoy. Those nucleic acids may be, for example, a nucleotide having a partial nucleotide sequence of continuous 14 bases or more in the nucleotide sequence of SEQ ID NO: 1 (sense chain or antisense chain) or a sequence complemental thereto.

The protein or polypeptide of the present invention or a protein or polypeptide having immunological equivalence thereto (e.g. a segment of the protein or a synthetic polypeptide having a partial sequence of the peptide) can be used as an antigen, and thereby there can be obtained an antibody which can recognize the protein or polypeptide of the present invention. The wording “having immunological equivalence” means that those cross-react with an antibody to the protein or polypeptide of the present invention.

The polyclonal antibody can be prepared by a conventional method of inoculating an antigen to a host animal (e.g. rats, rabbits. etc.) and collecting the immunized serum. The monoclonal antibody can be prepared by a conventional technique such as a hybridoma method. The monoclonal antibody may be converted into a humanized monoclonal antibody by modifying the gene thereof by a conventional method.

By using the antibodies as obtained above, the expression of the protein or polypeptide of the present invention in tissues or cells can be detected by a conventional immunochemical assay. The protein or polypeptide of the present invention may be purified by affinity chromatography using the above antibodies. The function of activity of the protein or polypeptide of the present invention may be modified (e.g. depressed) by using a neutralizing antibodies.

The receptor protein or polypeptide has a function or activity (biological activity) as a purinoceptor (P2Y receptor). The function or activity include the following activities.

(i) Specific binding properties to ATP, ADP or their analogues (e.g. 2MeSATP and 2MeSADP).

(ii) Induction of intracellular signal transduction based on stimulation by ATP, ADP or their analogues (e.g. 2MeSATP and 2MeSADP).

(iii) Activation of G protein [more specifically, G_{iα} (α subunit of G protein belonging to Gᵢ subfamily, e.g. G_{iα})] based on stimulation by ATP, ADP or their analogues (e.g. 2MeSATP and 2MeSADP).

The function or activity of the receptor protein or polypeptide of the present invention can be detected, for example, in the following manner.

Method for detecting the function or activity of (i):

The receptor protein or polypeptide of the present invention (in the form of a membrane fraction containing the protein or polypeptide, or in the form of cells on which surface the protein or polypeptide is expressed) is contacted to a ligand compound (e.g. ATP, ADP or analogues thereof such as 2MeSATP, 2MeSADP, etc.), and thereby the specific binding of both is detected. The detection of said binding can be carried out by using a labeled ligand compound (e.g. labeled with RI or fluorescence). The specific binding can be detected by a conventional competitive assay using the labeled ligand compound together with a non-labeled ligand compound.

Method for detecting the function or activity of (ii):

Cells on which surface the receptor protein or polypeptide of the present invention is expressed are contacted to a ligand compound (e.g. ATP, ADP or analogues thereof such as 2MeSATP, 2MeSADP, etc. which act as an agonist), and the thus-induced intracellular signal transduction (e.g. change of concentration of Ca²⁺, change of concentration of cAMP, activation of phospholipase C, change of pH value, change of concentration of K⁺, etc.) is detected. By using as a reference cells on which surface the receptor protein or polypeptide of the present invention is expressed in a lower level or is not expressed, the intracellular signal transduction is measured, and the intracellular signal transduction in the cells on which is expressed the receptor protein or polypeptide of the present invention is compared with that in the reference cells. When the level of the intracellular signal transduction is higher in the cells on which is expressed the receptor protein or polypeptide of the present invention than in the reference cells, it is evaluated that it has the function or activity of (ii) by the degree of the higher level.

The detection of the intracellular signal transduction is carried out in a similar manner as specifically disclosed in literatures, for example, the method of Chen et al., Analytical Biochemistry, Vol. 226, pp. 349-354, 1995 (change of concentration of Ca²⁺, change of concentration of cAMP); the method of Graminski et al., J. Biol. Chem., Vol. 268, pp. 5957-5964, 1993 (activation of phospholipase C); the method of Sakurai et al., Cell, Vol. 92, pp. 573-585, 1998 (change of concentration of Ca²⁺); the method of Hollopeter et al., Nature, Vol. 409, pp. 202-207, 2001 (change of concentration of K⁺); the method of Tatemoto et al., Biochem. Biophys. Res. Commun., Vol. 251, pp. 471-476, 1998 (change of pH value); the method of Hinuma et al., Nature, Vol. 393, pp. 272-273, 1998 (arachidonic acid metabolite releasing); JP-A-9-268, etc.

Method for detecting the function or activity of (iii):

From the cells on which membrane a fusion protein of the receptor protein or polypeptide of the present invention and G_{iα} (α subunit of G protein belonging to Gᵢ subfamily, e.g. G_{iαl}) is expressed, a membrane fraction is prepared. The membrane fraction is contacted with a labeled GTP or analogues thereof (e.g. a hardly hydrolizable GTP analogue, such as GTPγS (guanosine 5′-O-(3-thio-triphosphate), etc.) in the presence or absence of a ligand compound (e.g. ATP, ADP or analogues thereof such as 2MeSATP, 2MeSADP, etc. which act as an agonist). Thereafter, the binding of the labeled GTP or an analogue thereof to the membrane fraction is detected. When the level of binding in the case of testing in the presence of the ligand compound is higher than the level of binding in the case of testing in the absence of the ligand compound, it is evaluated that it has the function or activity of (iii) by the degree of the higher level.

The amino acid sequence and nucleotide sequence of the G_{iα} (α subunit of G protein belonging to Gᵢ subfamily, e.g. G_{iαl}) and a gene thereof have already been known [Human G_{iαl} (³⁵¹Cys→Ile)/Bahia et al., Biochemistry, Vol. 37, pp. 11555-11562, 1998; Human G_{iαl}/Genbank/EMBL accession no. AF055013, PIR/SWISS-PROT accession no. P04898, etc.].

Accordingly, a DNA encoding G_{iα} can be selected and obtained by a PCR (polymerase chain reaction) method, a colony-hybridization method, a plaque hybridization method, or a combination thereof using the above known sequence information. The DNA encoding G_{iα} is bound at understream of the DNA encoding the receptor protein or polypeptide of the present invention, and the resultant is inserted into a vector containing an appropriate promoter to obtain an expression vector suitable for expressing the fusion protein. The expression vector for expressing the fusion protein is inserted into cells and thereby the desired fusion protein can be expressed.

The receptor protein or polypeptide of the present invention can be used for screening or identifying a ligand or modulator (agonist or antagonist) thereto.

The screening or identifying the ligand or modulator (agonist or antagonist) to the receptor protein or polypeptide of the present invention can be carried out by a method comprising a step of contacting the receptor protein or polypeptide of the present invention (in the form of a membrane fraction containing the protein or polypeptide, or in the form of cells on which surface the protein or polypeptide is expressed) to a test compound; and a step of detecting (1) the specific binding of the receptor protein or polypeptide to the test compound, (2) the intracellular signal transduction, or (3) the activation of G protein (G_{iα}), in the presence of the test compound.

In more detail, the screening or identifying the ligand, agonist and antagonist can each be carried out as follows.

(A) Method for Screening or Identifying the Ligand:

It can be done by (1) contacting the receptor protein or polypeptide of the present invention (in the form of a membrane fraction containing the protein or polypeptide, or in the form of cells on which surface the protein or polypeptide is expressed) to a test compound, (2) detecting the specific binding of the test compound to the receptor protein or polypeptide of the present invention and (3) determining whether the test compound has an ability of binding to the receptor protein or polypeptide, or determining strength of the ability.

The detection of the specific binding can be carried out by a conventional competitive assay using a known labeled ligand compound (e.g. labeled with RI or fluorescence) together with a non-labeled ligand compound.

The test compound (ligand) having the specific binding ability will highly possibly be useful as a modulator (agonist or antagonist.

(B) Method for Screening or Identifying the Agonist:

It can be done by (1) contacting the receptor protein or polypeptide of the present invention (in the form of a membrane fraction containing the protein or polypeptide, or in the form of cells on which surface the protein or polypeptide is expressed) to a test compound, (2) detecting the intracellular signal transduction or the activation of G protein (G_{iα}) in the presence (or absence) of the test compound, and (3) determining whether the test compound has an ability of inducing the intracellular signal transduction or the activation of G protein based on the stimulation of the receptor protein or polypeptide, or determining strength of the ability.

The detection of the intracellular signal transduction (e.g. change of concentration of Ca²⁺ or cAMP, activation of phospholipase C, change of pH value, change of concentration of K⁺, etc.) and the detection of the activation of G protein (G_{iα}) are carried out in the same manner as described hereinbefore.

(C) Method for Screening or Identifying the Antagonist:

It can be done by (1) contacting the receptor protein or polypeptide of the present invention (in the form of a membrane fraction containing the protein or polypeptide, or in the form of cells on which surface the protein or polypeptide is expressed) to a test compound, (2) detecting the function or activity of the receptor protein or polypeptide in the presence (or absence) of the test compound, and (3) determining whether the test compound has an ability of depressing the function or activity of the receptor protein or polypeptide of the present invention, or determining strength of the ability.

The function or activity of the receptor protein or polypeptide of the present invention include the functions and activities as (i), (ii) and (iii) as described hereinbefore. The detection of these functions and activities are carried out in the same manner as described above.

In the determination of the ability of the test compound, it is preferable to use an appropriate reference control. Such a reference control includes a determination in the absence of a test compound, or a determination using cells in which the receptor protein of the present invention is not expressed or is expressed in a lower level. In order to determine more precisely, such a reference control is used in a combination of a plural of methods.

The receptor protein or polypeptide of the present invention to be used for the screening or identifying a ligand or modulator (agonist or antagonist) may be in the form of a membrane fraction containing the receptor protein or polypeptide, or in the form of cells on which surface the receptor protein or polypeptide is expressed.

As the cells on which surface the receptor protein or polypeptide is expressed, there may be used cells in which the receptor protein or polypeptide is overexpressed, for example, cells into which a recombinant vector containing a nucleic acid encoding the receptor protein or polypeptide is introduced.

The host cells to be introduced with the recombinant vector include cells (e.g. mammalian cells, insectile cells) on which membrane a foreign receptor protein can be expressed without losing its function. The host cells before introducing the recombinant vector are preferably to be ones not expressing the receptor protein or polypeptide to be introduced, or to be ones expressing that merely in a lower level.

The membrane fraction containing the receptor protein or polypeptide may be prepared by fracturing cells on which membrane the receptor protein or polypeptide is expressed, and subjecting the fractured cells to fractionation with centrifugation, such as differential centrifugation or density gradient centrifugation. For example, a fractured cell solution is centrifuged at a low speed (about 500-3000 rpm) for a short period of time (usually for about one to 10 minutes), and the supernatant is further centrifuged at a higher speed (about 15,000-30,000 rpm) for about 30 to 120 minutes, and the precipitation thus obtained is used as the membrane fraction.

### EXAMPLES

The present invention is illustrated in more detail by the following examples, but it should not be construed to be limited thereto.

Unless specified otherwise, the procedures in the following examples are carried out by the method disclosed in “Molecular Cloning” (edited by Sambrook, J., Fritsch, E. F. and Maniatis, T., issued by Cold Spring Harbor Laboratory Press, in 1989), and when commercially available reagents or kits are used, they are used in accordance with the instructions.

### Example 1

Isolation of cDNA of Human TG0022 Gene:

(1) By using as a query sequence the amino acid sequence of human δ opioid receptor (PIR/SWISS-PROT accession no. P41143), homology search was done on human EST database in NCBI (National Center for Biotechnology Information) by tblastn (Altschul S F et al., J. Mol. Biol., Vol. 215, pp. 403-410, 1990). As a result, an EST clone having high homology “IMAGE; 1689643” (GenBank/EMBL accession no. AI090920) was found. It was assumed that this EST clone includes one large open reading frame in the nucleotide sequence thereof. As the amino acid sequence of the polypeptide encoded by the assumed open reading frame, the existence of a transmembrane region was predicted by SOSUI (a predicting system for determining membrane protein and transmembrane) (Hirokawa et al., Bioinformatics, Vol. 14, pp. 378-379, 1998). Besides, the homology with known G protein-coupled receptor was also analyzed. From those results, it is assumed that this EST clone includes transmembrane regions III to V of G protein-coupled receptor.

(2) Based on the nucleotide sequence of EST clone obtained in the above (1), a primer was designed, and by using said primer, cDNA fragments containing respectively further a 5′-region and a 3′-region were obtained from the above EST by 5′- and 3′-RACE (Rapid Amplification of cDNA Ends).

The 5′-RACE was carried out as follow.

The first PCR was done by using a commercially available cDNA library wherein an adapter sequence was jointed to human brain-origin cDNA (a tradename “Marathon-Ready cDNA Brain”, manufactured by CLONTECH) as a template for PCR (polymerase chain reaction).

In the above, a synthetic oligonucleotide comprising nucleotide sequence of SEQ ID No: 3 (designated as “Primer TG0022GSP1”, which corresponds to the region containing 165th to 195th nucleotides of EST clone “IMAGE 1689643”) was used as antisense primer, and further an oligonucleotide comprising a nucleotide sequence corresponding to the adapter moiety of the template cDNA (Primer AP1, manufactured by CLONTECH) was used as a sense primer.

A PCR reaction solution containing these primer and template cDNA (25 λl) [components: template cNDA (tradename “Marathon-Ready cDNA Brain”, manufactured by CLONTECH) 1 μl, sterilized water 19.5 μl, a PCR buffer (Advantage 2 PCR Buffer, manufactured by CLONTECH) 2.5 μl, deoxynucleotide solution (dATP, dCTP, dGTP and dTTP, each 10 mM) 0.5 μl, polymerase solution (Advantage 2 Polymerase Mix, manufactured by CLONTECH) 0.5 μl, sense primer (10 μM) 0.5 μl, antisense primer (10 μM) 0.5 μl] was prepared, and it was subjected to PCR [conditions: 94° C., 30 seconds, (94° C., 5 seconds→72° C., 4 minutes)×5 times, (94° C., 5 seconds→70° C., 4 minutes)×5 times, (94° C., 5 seconds→68° C., 4 minutes)×25 times, 68° C., 4 minutes].

By using the PCR solution thus obtained (1 μl) as a template, 2nd PCR was done. In said procedure, a synthetic oligonucleotide comprising nucleotide sequence of SEQ ID No: 4 (designated as “Primer TG0022NGSP1”, which corresponds to the region containing 197th to 222nd nucleotides of EST clone “IMAGE 1689643”) was used as antisense primer, and further an oligonucleotide comprising a nucleotide sequence corresponding to the adapter moiety of the template cDNA (Primer AP2, manufactured by CLONTECH) was used as a sense primer. The components of the PCR reaction solution and the reaction conditions are the same as described above.

The thus-obtained PCR product was subjected to agarose gel electrophoresis, and the bands were cut out and purified to give a cDNA fragment containing 5′-regions (about 600 bp) (hereinafter, referred to as “5′cDNA fragment”).

In the same manner as in 5′-RACE, the 3′-RACE was done, excepting that for the 1st PCR, a synthetic oligonucleotide comprising nucleotide sequence of SEQ ID No: 5 (designated as “Primer TG0022GSP2”, which corresponds to the region containing 373rd to 398th nucleotides of EST clone “IMAGE 1689643”) was used as sense primer, and an oligonucleotide comprising a nucleotide sequence corresponding to the adapter moiety of the template cDNA (Primer AP1, manufactured by CLONTECH) was used as antisense primer, and further that for the 2nd PCR, a synthetic oligonucleotide comprising nucleotide sequence of SEQ ID No: 6 (designated as “Primer TG0022NGSP2”, which corresponds to the region containing 342nd to 369th nucleotides of EST clone “IMAGE 1689643”) was used as sense primer, and an oligonucleotide comprising a nucleotide sequence corresponding to the adapter moiety of the template cDNA (Primer AP2, manufactured by CLONTECH) was used as antisense primer. The thus-obtained PCR product was subjected to agarose gel electrophoresis, and the bands were cut out and purified to give a cDNA fragment containing 3′-regions (about 1000 bp) (hereinafter, referred to as “3′cDNA fragment”).

Each of the 5′cDNA fragment (about 600 bp) and 3′cDNA fragment (about 1000 bp) obtained above were linked to a vector plasmid (pGEM-T Easy, manufactured by Promega Corporation) respectively, and by using the plasmids thus obtained, the nucleotide sequences of each cDNA fragment were determined. The nucleotide sequence was determined by a dideoxy method (Thermo Sequenase Cycle Sequencing Kit, manufactured by USB) using an automatic DNA sequencer (Li-COR LIC-4200L(S)-2 DNA Analysis System, manufactured by Aroka) (hereinafter, the same method was applied).

As a result, the nucleotide sequence of SEQ ID NO: 7 was obtained as the sequence of 5′cDNA fragment, and the nucleotide sequence of SEQ ID NO: 8 was obtained as the sequence of 3′cDNA fragment (These contained any sequence of neither the primer nor the adapter).

In these sequences, one open reading frame was identified. As to the amino acid sequence of this open reading frame, the transmembrane region was predicted by SOSUI, and further homology with known G protein-coupled receptor was analyzed. As a result, it was assumed that the 5′cDNA (about 600 bp) contained the corresponding region of from the initiation codon to the transmembrane region IV of the novel G protein-coupled receptor, and 3′cDNA fragment (about 1000 bp) contained the corresponding region of from the transmembrane region IV to the stop codon of said receptor.

(3) Based on the information of the nucleotide sequences of cDNA obtained in the above (2), a primer was designed, and using it, a cDNA containing whole coding region was isolated by PCR as follows.

A commercially available human brain-origin cDNA (a tradename “Marathon-Ready cDNA Brain”, manufactured by CLONTECH) as used in the above (2) was used as a template for PCR. As a sense primer, an oligonucleotide comprising nucleotide sequence of SEQ ID NO: 9 (“Primer TG0022C501”) was used, and as an antisense primer, an oligonucleotide comprising a nucleotide sequence of SEQ ID NO: 10 (“Primer TG0022C301”) was used. The PCR reaction solution was the same as used in the above (2). Besides, the conditions of PCR were 94° C., 30 seconds, (94° C., 5 seconds→68° C., 3 minutes)×35 times, 68° C., 3 minutes.

The thus-obtained PCR product was subjected to agarose gel electrophoresis, and the bands were cut out and purified to give a cDNA fragment (about 1300 bp). This product is linked to a vector-plasmid (pGEM-T Easy, manufactured by Promega Corporation), and by using the plasmid (designated as “p186-1”), the nucleotide sequence of the cDNA was determined.

As a result, there was obtained the nucleotide sequence (1255 bp) as shown in SEQ ID NO: 1. When this nucleotide sequence was subjected to alignment with those of SEQ ID NO: 7 and SEQ ID NO: 8, it was completely identical with that of SEQ ID NO: 7 but was different from that of SEQ ID NO: 8 in one nucleotide. It was assumed that it would be due to genetic polymorphism or due to an error in PCR.

In the sequence of SEQ ID NO:1, one open reading frame was identified. The protein encoded thereby (designated as “TG0022 protein”) has an amino acid sequence (333 amino acid residues) as shown in SEQ ID NO: 2. As to said SEQ ID NO: 1 and SEQ ID NO: 2. the transmembrane region was predicted by SOSUI and further their homology with known protein-coupled receptor was analyzed. As a result, it was assumed that the TG0022 protein is a novel G protein-coupled receptor. It was also assumed that the cDNA (about 1300 bp) obtained above is a cDNA of a gene of TG0022 protein (designated as “TG0022 gene”) containing full length of the coding region.

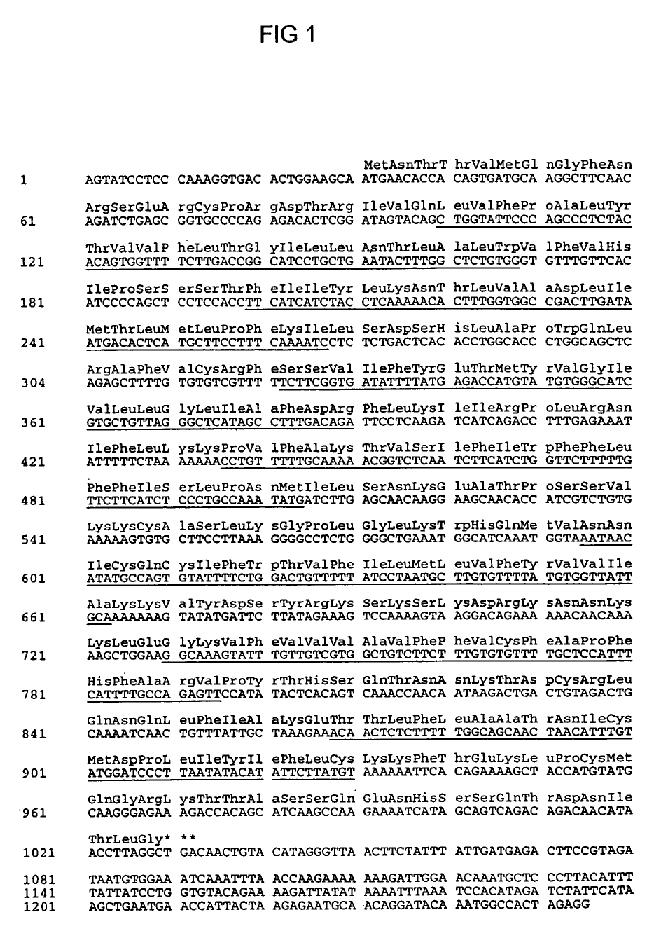

The accompanying FIG. 1 shows their nucleotide sequence and amino acid sequence and also the transmembrane regions (the underlined regions).

By blast method (Altschul et al., J. Mol. Biol., Vol. 215, pp. 403-410, 1990), the homology in amino acid sequence was analyzed. As a result, there have found two G protein-coupled receptors having a homology with TG0022 protein in some extent, i.e. P2Y₁₂ (platelet ADP receptor: Hollopeter et al., Nature, Vol. 409, pp. 202-207, 2001; NCBI accession no. AAG48944 and NP_{—}073625) and KIAA0001 (UDP-glucose receptor: PIR/SWISS-PROT accession no. Q15391). The homology in amino acid sequence was 45.3% between TG0022 protein and P2Y₁₂, and 43.2% between TG0022 protein and KIAA0001.

### Example 2

Expression Distribution of TG0022 Gene:

The distribution of expression of TG0022 gene in various tissues and cells in human was analyzed by dot blotting method. The dot blotting was done by using mRNA of human tissues and human culture cells (dotted on nylon membrane)(Multiple Tissue Expression Array, manufactured by CLONTECH) and RI-labeled probe. The membrane was dotted (trapped and fixed) with mRNAs [poly(A)RNA] originated from various human tissues and human cell lines.

The RI-labeled probe was prepared as follows. That is, the plasmid containing cDNA obtained in the above Example 1(3) was treated by restriction enzyme NotI, and subjected to agarose electrophoresis to give a DNA fragment having about 1300 bp (cDNA fragment containing full length of the coding region of TG0022 gene). By using this cDNA fragment as a template, labeling was done with a labeling kit (Prime-a-Gene Labeling system, manufactured by Promega Corporation) which contains a random primer (hexadeoxyribonucleotides), nucleotide mixture (dATP, dGTP and dTTP) and DNA polymeraze I (Large (Klenow)) Fragment) and [α-32P]dCTP, and thereafter, it was purified by gel filtration to give a RI-labeled probe.

The hybridization of the dot blot was done as follows.

The membrane fixed with mRNA was pre-incubated at 68° C. for 30 minutes in Solution I, said solution being prepared by heating Salmon Testes DNA (9.4 μg/μl: manufactured by SIGMA) (160 μl) at 97° C. for 5 minutes, cooling it in ice-water and then mixing with ExpressHyb hybridization Solution (manufactured by CLONTECH) (15 ml) which was previously warmed to 60° C.

Then, the membrane was hybridized at 65° C. for 12 hours in a labeled probe-containing hybridization solution, said solution being prepared by mixing the above RI-labeled probe (20 μl), human COT-1 DNA (1 μg/μl: manufactured by Roche; 30 μl), Salmon Testes DNA (9.4 μg/μl: manufactured by SIGMA; 16 μl), 20×SSC (3M sodium chloride, 300 mM sodium citrate, pH 7.0; 50 μl), and Nuclease Free H₂O (manufactured by Promega Corporation; 84 μl) to give a solution (200 μl), heating the solution at 97° C. for 5 minutes, and further incubating the solution at 68° C. for 30 minutes and then adding thereto the above-prepared Solution I (5 ml). Thereafter, the membrane was pre-washed with a 1% SDS-containing 2×SSC (300 mM sodium chloride, 30 mM sodium citrate, pH 7.0) at 65° C. for 20 minutes (5 times), and then washed with a 0.5% SDS-containing 0.1×SSC (15 mM sodium chloride, 1.5 mM sodium citrate, pH 7.0) at 55° C. for 20 minutes (two times).

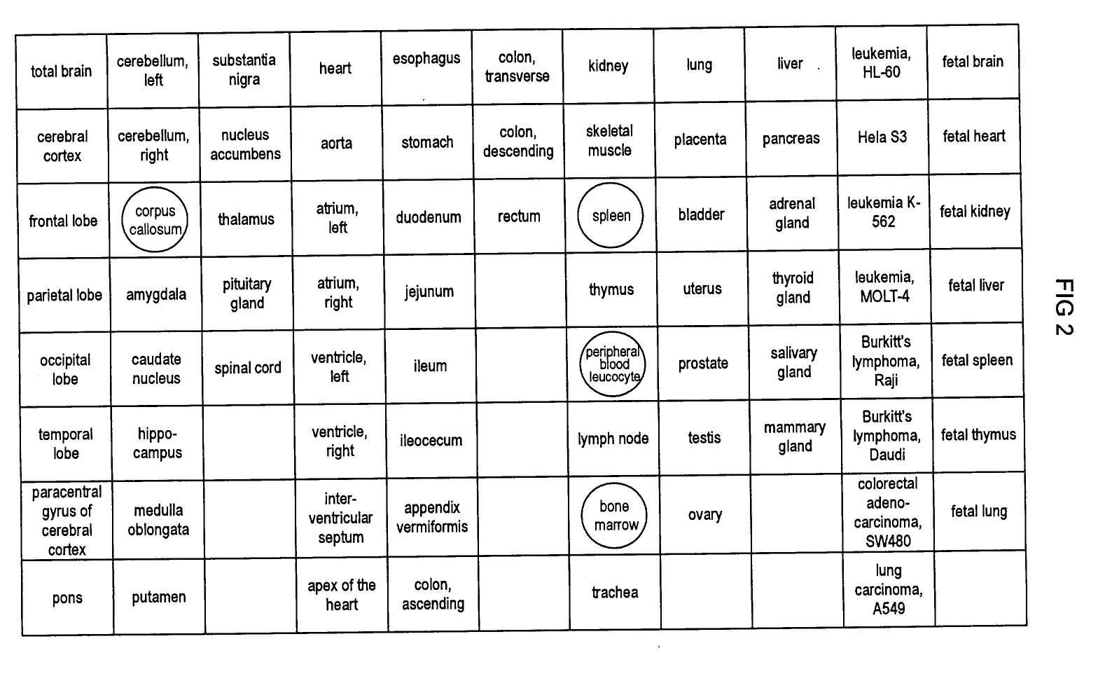

The signals of the hybridization was detected with an image analyzer (Bio-imaging Analysis System 2000, manufactured by Fuji Photo Film). As the results (as shown in FIG. 2), strong signals were observed in mRNA of the spleen, the peripheral blood leucocyte and the bone marrow, and further weak signal was observed in mRNA of the corpus callosum. From these results, it is assumed that the TG0022 gene will be expressed in the spleen, the peripheral blood leucocyte and the bone marrow, and is also expressed in the corpus callosum while somewhat weak.

### Example 3

Identification of Ligand of TG0022 Protein (G Protein-Coupled Receptor):

It was assumed that TG0022 protein would be a G protein-coupled receptor as is disclosed in Example 1. Then, by using a membrane fraction containing a fusion protein of TG0022 protein and G protein, the identification of ligand was done by a method of detecting a ligand-dependent binding with GTPγS (guanosine 5′-O-(3-thiotriphosphate)) (Wenzel-Seifert et al., Mol. Pharmacol., Vol. 58, pp. 954-966, 2000; Bahia et al., Biochemistry, Vol. 37, pp. 11555-11562, 1998).

(1) Preparation of a Plasmid for Expressing the Fusion Protein:

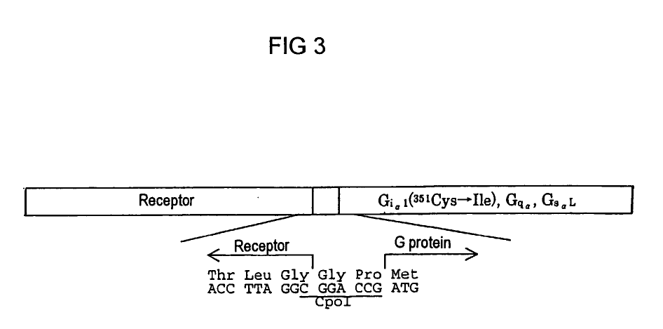

A plasmid for expressing a fusion protein of TG0022 protein and G protein [G_{1αl} (³⁵¹Cys→Ile), G_{qα} or G_{sαL}] as described in the following (i) to (iv). The outline of the fusion protein is shown in FIG. 3, wherein the G_{iαl} (³⁵¹Cys→Ile) means an isotype G_{iαl} derived by converting the 351st cysteine residue of G_{iαl} to isoleucine residue.

(i) Preparation of TG0022 Gene cDNA:

By using the plasmid containing the TG0022 gene cDNA (containing full length of the coding region) obtained in the above Example 1(3) as a template, the PCR was carried out, wherein synthetic oligonucleotides having the nucleotide sequences of SEQ ID NO: 11 and SEQ ID NO: 12 were used as a sense primer (Primer TG0022-1) and antisense primer (Primer TG0022-2), respectively.

These primers were designed on the basis of the nucleotide sequence (SEQ ID NO: 1) of cDNA of TG0022 gene so as to give as a PCR product a DNA fragment comprising cDNA encoding full length of TG0022 protein (excluding stop codon) and having at both termini recognition sites of restriction enzymes (NotI site and CpoI site).

The thus-obtained PCR product was ligated to a vector plasmid (a vector system for cloning a PCR product) (pGEM-T Easy Vector, manufactured by Promega Corporation), and the resulting plasmid was treated with restriction enzymes Not I and CpoI and the DNA fragment (about 1000 bp) was recovered.

(ii) Preparation of a Plasmid for Expressing a Fusion Protein with G Protein (G_{iαl} (³⁵¹Cys→Ile)):

PCR was carried out by using a human brain-origin cDNA (Marathon-Ready cDNA Brain, manufactured by CLONTECH) as a template, wherein synthetic oligonucleotides having the nucleotide sequences of SEQ ID NO: 13 and SEQ ID NO: 14 were used as a sense primer (Primer G_{iαl}-1) and antisense primer (Primer G_{iαl}-2), respectively.

These primers were designed on the basis of the known nucleotide sequence of cDNA encoding G_{iαl} protein (Genbank/EMBL accession no. AF055013) so as to give as a PCR product a DNA fragment comprising cDNA encoding full length of G_{iαl} protein.

The second PCR was carried out by using the PCR product prepared above as a template, wherein synthetic oligonucleotides having the nucleotide sequences of SEQ ID NO: 15 and SEQ ID NO: 16 were used as a sense primer (Primer G_{iαl}-3) and antisense primer (Primer G_{iαl}-4), respectively.

These primers were designed so as to give as a PCR product a DNA fragment comprising cDNA encoding full length of the G_{iαl}(³⁵¹Cys→Ile) (i.e. an isotype G_{iαl} protein derived by converting the 351st cysteine residue of G_{iαl} protein to isoleucine residue) and having at both termini recognition sites of restriction enzymes (CpoI site and BamHI site).

The thus-obtained PCR product was ligated to a vector plasmid (pGEM-T Easy Vector, manufactured by Promega Corporation). The plasmid was treated with restriction emzymes NotI and BamHI, and the resulting DNA fragment (about 1100 bp) was inserted into the NotI/BamHI sites of a vector plasmid: pBluescriptII (manufactured by Toyobo Co. Ltd.). Then, the DNA fragment prepared in the above (i) was inserted into the above plasmid at the NotI/CpoI sites to give a plasmid BSKII/G_{iαl} (³⁵¹Cys→Ile). This plasmid was treated with NotI and EcoRI and the thus produced DNA fragment (about 2100 bp) was recovered.

The DNA fragment was inserted into the NotI/EcoRI sites of baculovirus vector plasmid pVL1392 (manufactured by PharMingen) to give a plasmid for expression of a fusion protein: pVL1392/TG0022-G_{iαl} (³⁵¹Cys→Ile) (it may optionally be referred to as “Plasmid 158-15”).

(iii) Preparation of a Plasmid for Expressing a Fusion Protein with G Protein (G_{qα}):

PCR was carried out by using a human prostate-origin cDNA (Marathon-Ready cDNA Prostate, manufactured by CLONTECH) as a template, wherein synthetic oligonucleotides having the nucleotide sequences of SEQ ID NO: 17 and SEQ ID NO: 18 were used as a sense primer (Primer G_{qα}-1) and antisense primer (Primer G_{qα}-2), respectively.

These primers were designed on the basis of the known nucleotide sequence of cDNA encoding G_{qα} (Genbank/EMBL accession no. U43083) so as to give as a PCR product cDNA encoding full length of G_{qα}.

The second PCR was carried out by using the PCR product prepared above as a template, wherein synthetic oligonucleotides having the nucleotide sequences of SEQ ID NO: 19 and SEQ ID NO: 20 were used as a sense primer (Primer G_{qα}-3) and antisense primer (Primer G_{qα}-4) respectively. These primers were designed so as to give as a PCR product a DNA fragment comprising cDNA encoding full length of the G_{qα} and having at both termini recognition sites of restriction enzymes (CpoI site and BamHI site).

The thus-obtained PCR product was ligated to a vector plasmid (pGEM-T Easy Vector, manufactured by Promega Corporation). The plasmid was treated with restriction emzymes NotI and BamHI, and the resulting DNA fragment (about 1100 bp) was inserted into the NotI/BamHI sites of a baculovirus vector plasmid pVL1392 to give a plasmid pVL¹³⁹²/G_{qα}.

Then, the DNA fragment prepared in the above (i) was inserted into the above plasmid at the restriction enzyme NotI/CpoI sites to give a plasmid for expression of a fusion protein: pVL1392/TG0022-G_{qα} (it may optionally be referred to as “Plasmid 179-9”).

(iv) Preparation of a Plasmid for Expressing a Fusion Protein with G Protein (G_{sαL}):

PCR was carried out by using a human bone marrow-origin cDNA (Marathon-Ready cDNA Bone Marrow, manufactured by CLONTECH) as a template, wherein synthetic oligo-nucleotides having the nucleotide sequences of SEQ ID NO: 21 and SEQ ID NO: 22 were used as a sense primer (Primer G_{sαL}-1) and antisense primer (Primer G_{sαL}-2), respectively.

These primers were designed on the basis of the known nucleotide sequence of cDNA encoding G_{sαL} (Genbank/EMBL accession no. X04408) so as to give as a PCR product a DNA fragment comprising cDNA encoding full length of G_{sαL} protein and having at both termini recognition sites of restriction enzymes (CpoI site and XbaI site).

The thus-obtained PCR product was ligated to a vector plasmid (pGEM-T Easy Vector, manufactured by Promega Corporation). The plasmid was treated with restriction emzymes NotI and XbaI, and the resulting DNA fragment (about 1200 bp) was inserted into the NotI/XbaI sites of baculovirus vector plasmid pVL1392 (manufactured by PharMingen) to give a plasmid pVL1392/G_{sαL}.

Then, the DNA fragment prepared in the above (i) was inserted into the above plasmid at the restriction enzyme NotI/CpoI sites to give a plasmid for expression of a fusion protein: pVL1392/TG0022-G_{sαL} (it may optionally be referred to as “pTG0022-G_{sαL}/pVL1392-1”).

(2) Preparation of a Membrane Fraction Containing a Fusion Protein:

The three expression plasmids obtained in the above (1)-(ii) to -(iv) are a plasmid for expressing a fusion protein which has a structure as shown in the accormpanying FIG. 3 (schematic view) wherein TG0022 protein (full length) is bound to G protein [G_{iαl} (³⁵¹Cys→Ile), G_{qα} or G_{sαL}] via a linker sequence (-Gly-Pro-) bound to C-terminus of TG0022 protein.

These expression plasmids for fusion proteins, pVL1392/TG0022-G_{iα}ₗ (³⁵¹Cys Ile), pVL1392/TG0022-G_{qα or pVL}1392/TGoo22-G_{sαL} were expressed in insectile cells to prepare membrane fractions containing fusion proteins in the following manner.

Firstly, insectile cells Sf9 (Spodoptera frugiperda SF9) (manufactured by PharMingen) were seeded at about 60% confluency in 1.5 ml of a medium [Grace's Insect Cell Culture Medium (pH 6.2) manufactured by Lifetech Oriental, which contains 10% fetal bovine serum, 0.35 mg/ml sodium hydrogen carbonate, 3.3 mg/ml TC Yeastolate (manufactured by DIFCO), 3.3 mg/ml TC Lactalbumin Hydrolysate (manufactured by DIFCO), 0.1 mg/ml Streptomycin, and 100 units/ml Penicillin] in a 3 cm plate which was coated with collagen, and incubated at 27° C. for 15 minutes.

After removing the medium, a buffer for transfection (Transfection Buffer A, manufactured by PharMingen; 375 μl) was added to the vessel and thereto was added dropwise a previously prepared DNA solution (400 μl), said DNA solution being prepared by mixing the fusion protein expression plasmid (1 μg), Linearized BaculoGold Baculovilus DNA (manufactured by PharMingen; 0.125 μg), sterilized water (25 μl), incubating the mixture at 25° C. for 15 minutes, and adding thereto Transfection Buffer B (manufactured by PharMingen; 375 μl).

After cultivating the mixture at 27° C. for 4 hours, the culture broth was removed and thereto was added a medium (1.2 ml) and the mixture was cultivated at 27° C. for 5 days. The resulting culture broth was centrifuged (1,000×g 5 minutes), and the supernatant was taken to give Virus Solution I.

The Sf9 cells were inoculated in a 3 cm plate which was coated with collagen so as to be about 30% confluency, and thereto were added Virus Solution I obtained above (100 μl) and a medium (1.2 ml), and the mixture was cultivated at 27° C. for 4 days. The resulting culture broth was centrifuged (1,000×g, 5 minutes), and the supernatant was taken to give Virus Solution II.

The Sf9 cells were inoculated in a 10 cm plate which was coated with collagen so as to be about 70% confluency, and thereto were added Virus Solution II obtained above (500 μl) and a medium (12 ml), and the mixture was cultivated at 27° C. for 4 days. The resulting culture broth was centrifuged (1,000×g, 5 minutes), and the supernatant was taken to give Virus Solution III.

The Sf9 cells were inoculated in a 10 cm plate which was coated with collagen so as to be about 70% confluency, and thereto were added Virus Solution III obtained above (100 μl) and a medium (12 ml), and the mixture was cultivated at 27° C. for 4 days. The resulting cells were washed with a cooled PBS (phosphate buffered saline, pH 7.4) and suspended into a cooled dissolution buffer [20 mM Tris-HCl, pH 7.5, 1 mM EDTA, 0.2 mM phenylmethylsulfonyl fluoride, 10 μg/ml Pepstatin, 10 μg/ml Leupeptin, and 2 μg/ml Aprotinin; 3.6 ml], and then the mixture was homogenized with a Teflon homogenizer to fracture the cells. The fractured cells were centrifuged (600×g, 10 minutes), and the resulting supernatant was further centrifuged (50,000×g, 20 minutes). The resulting precipitates were suspended into a reaction buffer [20 mM Tris-HCl, pH 7.5, 100 mM sodium chloride, 10 mM magnesium chloride; 450 μl] with a Teflon homogenizer to give a membrane fraction wherein the fusion protein was expressed.

(3) Test for Binding the Fusion Protein-Containing Membrane Fraction with GTPγS:

The membrane fraction obtained in the above (2) (450 μl) was suspended into a reaction buffer (7.54 ml), and thereto was added GDP (10 mM, 10 μl). To the mixture (160 μl) was added a test sample (20 μl), and the mixture was incubated at 30° C. for 10 minutes, and thereto was added [³⁵S]GTPγS (5 nM, 5 nCi/μl: manufactured by Amersham Pharmacia Biotech; 20 μl), and then the reaction was initiated. After incubating at 30° C. for 1 hour, the reaction mixture was filtered with a glass filter (UniFilter-96 GF/B, manufactured by PACKHARD) to terminate the reaction. The filter was washed with a cooled reaction buffer (200 μl) three times, and the amount of [³⁵S]GTPγS (amount bound to the membrane fraction) on the filter was measured by a liquid scintillator. The amount of specifically bound [³⁵S] GTPγS was calculated by reducting an amount of non-specifically bound [³⁵S] GTPγS (which was measured in the presence of 10 μM GTPγS) from the amount of [³⁵S]GTPγS obtained above.

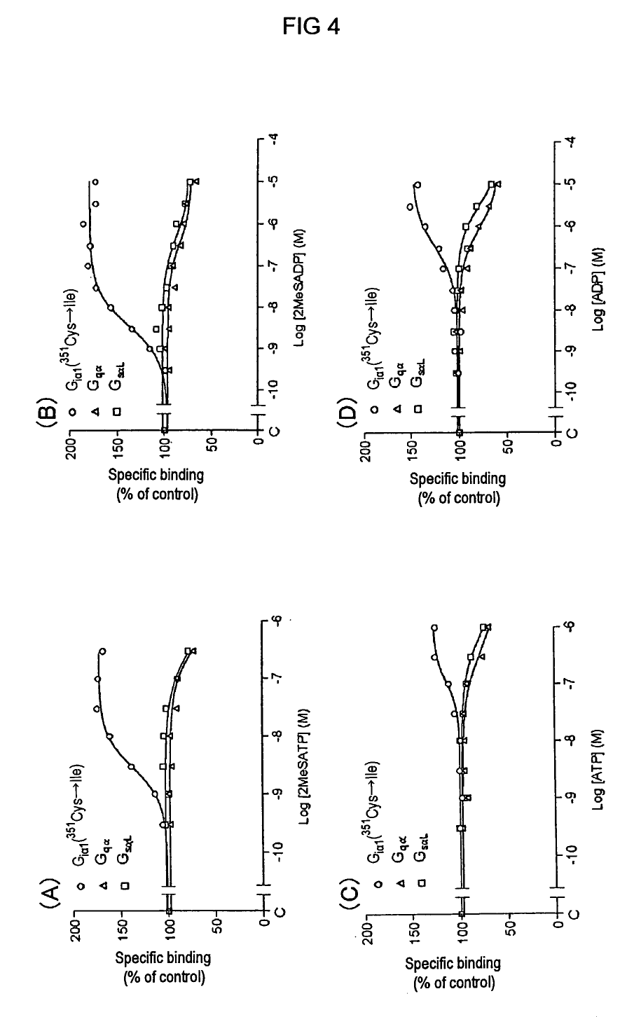

About 80 kinds of nucleic acid compounds were used as the test compounds. The results are shown in the accompanying FIG. 4. As is shown in the figures, in case of using a membrane fraction containing a fusion protein of TG0022 protein and G_{iαl} (³⁵¹Cys→Ile), the specific binding amount of [³⁵S] GTPγS was increased dependent on the concentration by adding 2MeSATP (2-methylthioadenosine 5′-triphosphate), 2MeSADP (2-methylthioadenosine 5′-diphosphate), ATP, and ADP.

On the other hand, in case of using a membrane fraction containing a fusion protein of TG0022 protein and G_{qαl} and a fusion protein of TG0022 protein and G_{sαL} any increase of the specific binding amount was observed even by addition of those compounds.

From these test results, it is assumed that the TG0022 protein is a G protein-coupled receptor where it conjugates to the G_{iαl} protein. TG0022 protein will be considered to be a purinoceptor for which 2MeSATP, 2MeSADP, ATP, and ADP function as a ligand (agonist).

### INDUSTRIAL APPLICABILITY

The receptor protein and gene thereof of the present invention are useful for research of mechanism of intracellular signal transduction, and further will be used as a target molecule for a new medicament of diseases.

Moreover, the method of screening, identifying and characterizing the agonist or antagonist using the receptor protein and gene thereof of the present invention will be useful for research and development of new medicaments.

1. A polypeptide having the function or activity of a purinoceptor which is selected from the following (A), (B) and (C):
(A) a polypeptide having an amino acid sequence of SEQ ID NO: 2,
(B) a polypeptide having an amino acid sequence shown by SEQ ID NO: 2 in which one or plural amino acids are deleted, substituted or added,
(C) a polypeptide encoded by a nucleic acid or a complement thereof, said nucleic acid being hybridizable under stringent conditions to the nucleic acid having the nucleotide sequence of SEQ ID NO: 1.

2. The polypeptide as claimed in claim 1, wherein the purinoceptor is P2Y receptor.

3. The polypeptide as claimed in claim 1, which is a polypeptide of human.

4. The polypeptide as claimed in claim 1, which is a polypeptide of a mammal.

5. The polypeptide as claimed in claim 1, wherein the function or activity of a purinoceptor is selected from the following (i), (ii) and (iii):
(i) specific binding properties to ATP, ADP or their analogues,
(ii) induction of intracellular signal transduction based on stimulation by ATP, ADP or their analogues,
(iii) activation of G protein based on stimulation by ATP, ADP or their analogues.

6. The polypeptide as claimed in claim 1, wherein the function or activity of a purinoceptor is selected from the following (i), (ii) and (iii):
(i) specific binding properties to ADP or analogues thereof,
(ii) induction of intracellular signal transduction based on stimulation by ADP or analogues thereof,
(iii) activation of G protein based on stimulation by ADP or analogues thereof.

7. A nucleic acid encoding the polypeptide as claimed in claim 1.

8. A nucleic acid encoding a polypeptide having the function or activity of a purinoceptor which is selected from the following (a) or (b):
(a) a nucleic acid having a nucleotide sequence of SEQ ID NO: 1,
(b) a nucleic acid or a complement thereof, said nucleic acid being hybridizable under stringent conditions to the nucleic acid having the nucleotide sequence of SEQ ID NO: 1.

9. The nucleic acid as claimed in claim 8, wherein the purinoceptor is P2Y receptor.

10. The nucleic acid as claimed in claim 8, which is a nucleic acid of human.

11. The nucleic acid as claimed in claim 8, which is a nucleic acid of a mammal.

12. The nucleic acid as claimed in claim 8, wherein the function or activity of a purinoceptor is selected from the following (i), (ii) and (iii):
(i) specific binding properties to ATP, ADP or their analogues,
(ii) induction of intracellular signal transduction based on stimulation by ATP, ADP or their analogues,
(iii) activation of G protein based on stimulation by ATP, ADP or their analogues.

13. The nucleic acid as claimed in claim 8, wherein the function or activity of a purinoceptor is selected from the following (i), (ii) and (iii):
(i) specific binding properties to ADP or analogues thereof,
(ii) induction of intracellular signal transduction based on stimulation by ADP or analogues thereof,
(iii) activation of G protein based on stimulation by ADP or analogues thereof.

14. A recombinant vector containing the nucleic acid as claimed in claim 8.

15. A host cell introduced with the recombinant vector as defined in claim 14.

16. A method for detecting a function or activity of the polypeptide as claimed in claim 1, said function or activity being selected from the following (i), (ii) and (iii):
(i) specific binding properties to ATP, ADP or their analogues,
(ii) induction of intracellular signal transduction based on stimulation by ATP, ADP or their analogues,
(iii) activation of G protein based on stimulation by ATP, ADP or their analogues.

17. The method as claimed in claim 16, wherein the analogue of ATP or ADP is 2-methylthioadenosine 5′-triphosphate (2MeSATP) or 2-methylthioadenosine 5′-diphosphate (2MeSADP).

18. The method as claimed in claim 16, wherein the intracellular signal transduction is selected from the change of concentration of Ca²⁺, the change of concentration of cAMP, activation of phospholipase C, change of pH value, and change of concentration of K⁺.

19. The method as claimed in claim 16, wherein the activation of G protein is an activation of a subunit of G protein belonging to Gᵢ subfamily.

20. The method as claimed in claim 16, wherein the polypeptide defined in claim 1 is in the form of a membrane fraction containing the polypeptide, or in the form of cells on which surface the polypeptide is expressed.

21. The method as claimed in claim 20, wherein the cells on which surface the polypeptide is expressed are cells in which the polypeptide is overexpressed by introducing a nucleic acid encoding the polypeptide or an expression vector containing the nucleic acid.

22. A method for enhancing the function or activity of the polypeptide as defined in claim 1, which comprises enhancing the expression of the polypeptide in cells.

23. The method as claimed in claim 22, which comprises introducing the nucleic acid as defined in claim 7 or 8 or a recombinant vector containing the nucleic acid into cells.

24. A method for depressing the function or activity of the polypeptide as defined in claim 1, which comprises introducing an oligonucleotide or a complement thereof into cells, said oligonucleotide being hybridizable under stringent conditions to the nucleic acid having the nucleotide sequence of SEQ ID NO: 1.

25. A method for depressing the function or activity of the polypeptide as defined in claim 1, which comprises contacting an antibody recognizing the polypeptide to cells.

26. The method as claimed in claim 25, wherein the antibody is an antibody which neutralizes the function or activity of the polypeptide as defined in claim 1.

27. The method of any one of claims 22 to 26, wherein the function or activity of the polypeptide as defined in claim 1 is selected from the following (i), (ii) and (iii):
(i) specific binding properties to ATP, ADP or their analogues,
(ii) induction of intracellular signal transduction based on stimulation by ATP, ADP or their analogues,
(iii) activation of G protein based on stimulation by ATP, ADP or their analogues.

28. A method for screening or identifying a ligand, agonist or antagonist to the polypeptide as defined in claim 1, which comprises
a step of contacting the polypeptide to a test compound; and
a step of detecting (1) the specific binding of the receptor protein or polypeptide to the test compound, (2) the intracellular signal transduction, or (3) the activation of G protein, in the presence of the test compound.

29. The method as claimed in claim 28, wherein the intracellular signal transduction is selected from the change of concentration of Ca²⁺, the change of concentration of cAMP, activation of phospholipase C, change of pH value, and change of concentration of K⁺.

30. The method as claimed in claim 28, wherein the activation of G protein is an activation of a subunit of G protein belonging to Gᵢ subfamily.

31. A method for screening or identifying a ligand to the polypeptide as defined in claim 1, which comprises
(1) a step of contacting the polypeptide to a test compound;
(2) a step of detecting the specific binding of the test compound to the polypeptide; and
(3) a step of determining whether the test compound has an ability of binding to the polypeptide, or the strength of said ability.

32. A method for screening or identifying an agonist to the polypeptide as defined in claim 1, which comprises
(1) a step of contacting the polypeptide to a test compound;
(2) a step of detecting the intracellular signal transduction or the activation of G protein in the presence of the test compound; and
(3) a step of determining whether the test compound has an ability of inducing the intracellular signal transduction or the activation of G protein based on the stimulation of the polypeptide, or the strength of said abilitiy.

33. A method for screening or identifying an antagonist to the polypeptide as defined in claim 1, which comprises
(1) a step of contacting the polypeptide to a test compound;
(2) a step of detecting the function or activity of the polypeptide in the presence of the test compound; and
(3) a step of determining whether the test compound has an ability of depressing the function or activity of the polypeptide, or the strength of said ability.

34. The method as claimed in claim 33, wherein the function or activity of the polypeptide as defined in claim 1 is selected from the following (i), (ii) and (iii):
(i) specific binding properties to ATP, ADP or their analogues,
(ii) induction of intracellular signal transduction based on stimulation by ATP, ADP or their analogues,
(iii) activation of G protein based on stimulation by ATP, ADP or their analogues.

35. The method as claimed in claim 33, wherein the function or activity of the polypeptide as defined in claim 1 is selected from the following (i), (ii) and (iii):
(i) specific binding properties to ADP or analogues thereof,
(ii) induction of intracellular signal transduction based on stimulation by ADP or analogues thereof,
(iii) activation of G protein based on stimulation by ADP or analogues thereof.

36. The method of any one of claims 28 to 35, wherein the polypeptide defined in claim 1 is in the form of a membrane fraction containing the polypeptide, or in the form of cells on which surface the polypeptide is expressed.

37. The method as claimed in claim 36, wherein the cells on which surface the polypeptide is expressed are cells on which the polypeptide is overexpressed by introducing a nucleic acid encoding the polypeptide or an expression vector containing the nucleic acid.
